(19) United States
(12) Patent Application Publication
(10) Pub. No.: US 2013/0059320 A1
(43) Pub. Date: Mar. 7, 2013

(54) Selective growth media for campylobacter bacteria and plating media with said growth media

(71) Applicant: Lawrence Restaino, Elburn, IL (US)

(72) Inventor: Lawrence Restaino, Elburn, IL (US)

(21) Appl. No.: 13/573,210

(22) Filed: Aug. 30, 2012

Related U.S. Application Data
(60) Provisional application No. 61/575,795, filed on Aug. 30, 2011.

(51) Int. Cl. G01N 21/78; C12N 1/20

(52) U.S. Cl. 435/19; 435/253.6

(57) ABSTRACT

A selective growth media for Campylobacter bacteria comprising nutrients, growth inhibitors for organisms other than Campylobacter including bile salts, Irgasan DP 300, sodium cefsulodin hydrate, cycloheximide and sodium cefoperazone, and one or more ingredients for absorbing oxygen. Also, selective plating media for Campylobacter bacteria comprising said growth media, a thickening agent and Aldol acetate.

The present invention relates to the field of selective growth media and to culture media for the detection and identification of bacteria using such selective growth media, and more particularly to such selective growth media and culture media for the detection and identification of Campylobacter bacteria.

### BACKGROUND OF THE INVENTION

Campylobacter bacteria are the cause of campylobacteriosis in humans and animals. Most people who become ill with campylobacteriosis get diarrhea, cramping, abdominal pain, and fever within two to five days after exposure to the organism. Campylobacteriosis generally requires a week to 10 days for the patient to recover.

Patients with such symptoms are common in the United States and foreign countries and Campylobacter is amoung the most prevelent causes of these symptoms. It has been estimated that campylobacteriosis affects over 2.4 million persons every year in the United States, or 0.8 % of the population. While campylobacteriosis seldom results in death or long term illness, treating the symptoms of the illness results in significant health costs to both government and private sector. Accordingly the need to care for patients with symptoms of campylobacteriosis requires prompt diagnosis, and the large number of patients requires test procedures to be conducted economically.

Campylobacteriosis is generally diagnosed by finding Campylobacter bacteria in association with the patient. Unfortunately the nature of the Campylobacter bacteria has impeded both a prompt diagnosis and an inexpensive diagnosis. Most Campylobacter related human illness is caused by the species Campylobacter jejuni or Campylobacter coli, but human illness can also be caused by other species. Campylobacter jejuni and Campylobacter coli are temperature sensitive, cannot tolerate drying and grow only in places with less oxygen than the amount in the atmosphere. As a result, culture media of the prior art have not only required long incubation times and have lacked specificity, but the colonies of Campylobacter jejuni and Campylobacter coli have appeared small on the incubated media of the prior art and difficult to locate against a background of more rapidly growing organisms.

### SUMMARY OF INVENTION

It is an object of the present invention to provide a culture medium that overcomes the prior art deficiencies for the detection and identification of the principle strains of Campylobacter that produce campylobacteriosis, namely Campylobacter jejuni and Campylobacter coli.

More specifically, it is an object of the present invention to provide a culture medium in which incubated plates exhibit fewer colonies of non-target or background organisms.

Further, it is an object of the present invention to provide a culture medium in which colonies of Campylobacter bacteria appear larger and brighter on incubated plates than on prior art incubated plates.

These objects are achieved by an agar plating media that contains (1 ) a substrate that reacts with the enzyme C-2 esterase, but substantially no other organism, to release precipitate of a distinctive color into the medium, and (2 ) a selective growth media for Campylobacter jejuni and Campylobacter coli bacteria containing nutrients and growth facilitators for Campylobacter bacteria to enlarge and accelerate growth of target colonies, and inhibitors for non-target organisms.

### DETAILED DESCRIPTION OF THE INVENTION

The selective growth medium of the present invention contains nutrients to promote growth of Campylobacter bacteria. The inventor has found that a selective growth medium achieving the objects of the present invention contains a mixture including one or more of the ingredients of the group heart infusion broth, vitamin mixture, lactalbumin hydrolysate, casamino acids, α-ketoglutaric acid and yeast extract. The preferred embodiment of the present invention described hereinafter utilizes a mixture of all of the ingredients of the foregoing group.

The inventor has found that one or more ingredients of the following group improve aero tolerance of the Campylobacter, when added to the mixture: hemin, sodium metabisulfite, sodium carbonate and sodium pyruvate. The preferred embodiment utilizes all of the ingredients of the foregoing group in its mixture.

Also selective growth media according to the present invention include one or more inhibitors of a group that the inventor has found are not capable of inhibiting the growth of Campylobacter bacteria. An inhibitor for gram positive bacteria is utilized, and the inventor has found bile salts #3 to be preferable. Other inhibitors of gram positive bacteria can also be used. The inventor has also found that Irgasan DP 300 is desirable in a selective growth medium and in a Campylobacter plating media according to the present invention materially reduces the background display on cultured plates, especially enteric bacteria such as Escherichia coli. Also, the inventor found it desirable for the selective growth media to contain sodium cefsulodin hydrate to inhibit Pseudomonas and Aeromonas type bacteria without affecting the growth of Campylobacter bacteria, and cycloheximide to prevent the growth of yeasts and molds. In addition, the inventor found that sodium cefoperazone is effective in selective growth media according to the present invention to inhibit the growth of other Campylobacter strains than Campylobacter jejuni or Campylobacter coli. The above described growth inhibitors form a group suitable for use in selective growth media for Campylobacter bacteria and one or more of the inhibitors of this group may be used in any particular medium, with or without any other inhibitor. Other growth inhibitors of non-target organisms that do not adversely effect the growth of Campylobacter bacteria may also be used.

The inventor has found that a selective growth medium suitable for use in Campylobacter plating media that achieves the objects of the present invention is achievable with at least all of the following growth inhibitors: cycloheximide, Irgasan DP 300, bile salts, sodium cefsuladin hydrate and cefoperazone sodium salt.

The inventor's preferred identification system for Campylobacter bacteria utilizes a solid plating medium containing a substrate that reacts with the enzyme C-2 esterase (acetate hydrolysis) which is released by Campylobacter bacteria but does not react with many other enzymes, thereby discriminating against bacteria that release such other enzymes. The preferred substrate is Aldol 515 acetate (A-4673_POO) which is produced by Biosynth A. G. of Switzerland under the trade name ALDOL. On contact with this substrate, Campylobacter jejuni and Campylobacter coli bacteria release enzyme that cleave the ALDOL substrate from the acetate group to produce a dark salmon precipitate into the medium, but other strains of Campylobacter that may grow on the medium do not.

The preferred plating medium comprises a mixture of the novel selective growth medium and the identification system, and this mixture is solidified by mixing Bacto-agar at 17 grams/liter to reduce the swarming of Campylobacter with the other ingredients of the medium. In addition, the inventor mixes titanium (IV) oxide into the combined mixture resulting in a white opaque medium which causes a white opaque background and results in a better contrast with the dark salmon Campylobacter colonies.

The preferred embodiment of the plating medium contains the ingredients in the proportions set forth in the following Table I.

TABLE 1

| | Supplier | Grams/Liter |
|---|---|---|
| Chemical | | |
| Heart Infusion Broth | Difco | 9.5 |
| Lactalbumin hydrolysate | Anyplace | 5.0 |
| Casamino acids | Anyplace | 4.189 |
| Yeast Extract | Anyplace | 4.5 |
| Hemin | Sigma | 0.01 |
| α-ketoglutaric acid | Anyplace | 1.0 |
| Cycloheximide | Anyplace | 0.2 |
| Irgasan DP 300 | Anyplace | 0.001 |
| Titanium (IV) oxide | Sigma | 3.0 |
| Bile Salts #3 | Fisher/VWR | 0.5 |
| Sodium metabisulfite (97+ %) | Anyplace | 0.5 |
| Sodium carbonate (anhydrous) | Anyplace | 0.6 |
| Sodium pyruvate | Anyplace | 1.0 |
| Vitamin Mixture | α-Bioscience C0607 | 2.5 |
| Bacto-agar | Difco | 17.0 |
| Supplements | | |
| Sodium cefsulodin hydrate | RPI | 0.006 |
| Cefoperazone, sodium salt | Sigma | 0.033 |
| Aldol 515 acetate, A4673_POO | Biosynth A.G. | 0.070 |
| | | (in 6 ml |
| | | of ethanol) |

Except for sodium cefsulodin hydrate, sodium cefoperazone Aldol 515 acetate, the ingredients are mixed in any order, the pH adjusted to 6.8 to 6.9, boiled to sterilize the mixture, and the mixture is permitted to cool to 48 -50 degrees Celsius. Thereafter, the supplements listed in Table 1, in sterile condition and at room temperature, are added aseptically to the other ingredients. The composition is then poured into plates and permitted to dry for 48 hours in the dark, and the plates are then ready to be used. Storage time of poured plates is as much as 45 days at 2 to 8 degrees Celsius. The final pH is between 6.8 and 7.2.

The process of the present invention requires a plate or mass of the plating medium to be inoculated with the test sample, and the inoculated mass is then incubated at 41°-42° Celsius for 48 hours under microaerophilic conditions to permit growth of the microorganisms in the test sample to observable dark salmon colonies 1-2 millimeters in diameter. The inventor has found that with the preferred plating medium described above, a period of 24 to 48 hours of incubation is sufficient time for Campylobacter colonies present in a test sample to grow into colonies that are readily observable with the naked eye. It is believed that the abundant growth of microorganisms in the preferred plating medium is due to the nutrients provided by the ingredients listed above and in Table 1. The surface of the plating medium mass is then assayed and the presence and number of dark salmon colonies, 1 to 2 millimeters in diameter with and without a clear ring, are recorded.

It is to be noted that no special equipment is required to observe the incubated mass of plating medium. The time required to note the number and presence of dark salmon colonies is far less than required when other colonies are present. Also, there are no ingredients in the plating medium that are especially costly. Hence, an assay of a test sample may be made at reduced cost from assays made with prior plating media.

Table 2 sets forth the results of tests made using the medium of Table 1 as described above and 188 microbial strains.

TABLE 2
TEST RESULTS FOR VARIOUS BACTERIAL STRAINS ON MEDIUM OF TABLE 1 INCUBATED
UNDER MICROAEROPHILIC CONDITIONS AT 41-42° C. FOR 48 HOURS

| | No. of | |
|---|---|---|
| Microbial Strains* | strains tested | Colonial Morphologies |
| Campylobacter jejuni | 54 | Flat to convex, dark salmon colonies; |
| | | 1.0 to 2.0 mm in diameter with and |
| | | without a clear ring and no swarming |
| Campylobacter coli | 38 | Flat to convex, dark salmon colonies; |
| | | 1.0 to 2.0 mm in diameter with and |
| | | without a clear ring and no swarming |
| Campylobacter lari | 5 | Flat to convex, beige to off white |
| | | colonies; 1.0 to 2.0 mm in diameter |
| | | with a clear ring and no swarming |
| Acinetobacter calcoaceticus | 1 | Convex, salmon colonies; <0.5 to 2.0 mm |
| | | in diameter |
| Acinetobacter baumannii | 1 | Scant growth; convex, salmon colonies; <0.5 |
| | | to 1.5 mm in diameter |
| Pseudomonas sp. | 1 | Flat, off white colonies; 2.0 mm in diameter |
| Enterobactar aerogenes | 1 | Scant growth; flat, white colonies; <1.0 mm |
| | | in diameter |
| Generic Escherichia coli | 1 | Scant growth; flat, white colonies; <1.0 mm |
| | | in diameter |
| Enterococcus faecalis | 1 | Scant growth; pinpoint white colonies; <1.0 mm |
| | | in diameter |
| Candida albicans | 1 | Convex, white colonies; 1.0 mm in diameter |
| Gram negatives & yeasts: Acinetobacter johnsonii, | 65 | No Growth |
| Acinetobacter grimontii, Acinetobacter sp., Aeromonas | | |
| hydrophila, Alcaligenes sp., Candida paludigena, | | |
| Citrobacter freundii(2), Escherichia vulneris, | | |
| Edwardsiella tarda, Enterobacter sp.(4), Escherichia coli | | |
| (3), Escherichia coli O157: H7(2), Flavobacterium sp.(3), | | |
| Hafnia alvei (2), Klebsiella pneumonia(2), Kluyvera | | |
| ascorbata, Moraxella sp.(4), Morganella morganii(2), | | |
| Ochrobactrum anthropi, Pantoea agglomerans, Proteus | | |
| mirabilis, Providencia alcafaciens, Pseudomonas sp.(9), | | |
| Rhodococcus rhodochrous, Saccharomyces cerevisiae, | | |
| Salmonella sp.(7), Serratia sp.(4), Shewanella | | |
| putrefacieni, Shigella sp.(2), Stenotrophomona | | |
| maltophilia, Vibrio parahaemolyticus, Yersinia | | |
| enterocolitica | | |
| Gram positives: Actinomyces viscosus, Bacillus sp.(3), | 19 | No Growth |
| Carnobacterium mobile, Corynebacterium matruchotii, | | |
| Enterococcus faecalis, Lactobacillus sp.(2), Leueonostac | | |
| mesenteroides, Listeria sp.(3), Micrococcus luteus, | | |
| Paenibacillus macerans, Salinicoccus hispanicus, | | |
| Staphylococcus aureus(2) and Streptococcus sp. | | |

*156 microbial strains tested by University of Guelph

The art will devise other uses for the selective growth media of the present invention including as a source of Campylobacter bacteria for laboratory study or experimentation, or use as a part of a plating media with a different detection system than that disclosed in this application. Likewise those skilled in the art may utilize the plating media of the present invention for uses not specifically described in the foregoing specification, but within the scope of the present invention. It is therefore intended that the scope of the present invention be not limited by the foregoing specification, but rather only by the appended claims.

The invention claimed is:

1. Isolation plating media for the presumptive identification of Campylobacter bacteria from a sample that may also contain other microorganisms, said medium being of a first color, comprising one or more nutrients for Campylobacter bacteria, one or more growth inhibitors for organisms other than Campylobacter, one or more ingredients for absorbing oxygen, a chromogenic substrate that reacts with C-2 esterace enzyme to produce a second color in the plating medium in the vicinity of the reaction that contrasts with the first color, and a sufficient mass of an agent to solidify the mixture, whereby microorganisms in the medium that produce C-2 esterace including Campylobacter bacteria produce colonies in the plating medium of the second color.

2. Isolation plating media comprising claim 1 wherein the one or more nutrients for Campylobacter bacteria consist of members of the group heart infusion broth, vitamin mixture, lactalbumin hydrolysate, casamino acids, α-ketoglutaric acid, and yeast extract.

3. Isolation plating media comprising claim 1 wherein the one or more ingredients for absorbing oxygen consist of members of the group hemin, sodium metabisulfite, sodium carbonate and sodium pyruvate.

4. Isolation plating media comprising claim 1 wherein one or more growth inhibitors for organisms other than Campylobacter consist of members of the group bile salts, Irgasan DP 300, sodium cefsulodin hydrate, cycloheximide and sodium cefoperazone.

5. Isolation plating media comprising claim 1 wherein the chromogenic substrate that reacts with C-2 esterace enzyme is Aldol acetate.

6. Isolation plating media for the presumptive identification of Campylobacter bacteria from a sample that may also contain other microorganisms, said medium being of a first color, comprising one or more nutrients for Campylobacter bacteria selected from the group heart infusion broth, vitamin mixture, lactalbumin hydrolysate, casamino acids, α-ketoglutaric acid and yeast extract, one or more growth inhibitors for organisms other than Campylobacter consist of members of the group bile salts, Irgasan DP 300, sodium cefsulodin hydrate, cycloheximide and sodium cefoperazone, one or more ingredients for absorbing oxygen, one or more growth inhibitors for organisms other than Campylobacter, one or more ingredients for absorbing oxygen consist of members of the group hemin, sodium metabisulfite, sodium carbonate and sodium pyruvate, a chromogenic substrate that reacts with C-2 esterace enzyme to produce a second color in the plating medium in the vicinity of the reaction that contrasts with the first color, and a sufficient mass of an agent to solidify the mixture, whereby microorganisms in the medium that produce C-2 esterace including Campylobacter bacteria produce colonies in the plating medium of the second color.

7. A selective growth media for Campylobacter bacteria from a sample that may also contain other microorganisms comprising one or more nutrients selected from the group heart infusion broth, vitamin mixture, lactalbumin hydrolysate, casamino acids, α-ketoglutaric acid and yeast extract, one or more growth inhibitors for organisms other than Campylobacter, and one or more ingredients for absorbing oxygen.

8. A selective growth media comprising claim 7 wherein the one or more ingredients for absorbing oxygen consist of members of group hemin, sodium metabisulfite, sodium carbonate and sodium pyruvate.

9. A selective growth media comprising claim 7 wherein the plurality of growth inhibitors for organisms other than Campylobacter consist of members of the group bile salts, Irgasan DP 300, sodium cefsulodin hydrate, cycloheximide and sodium cefoperazone.

10. A selective growth media for Campylobacter bacteria from a sample that may also contain other microorganisms comprising one or more nutrients selected from the group heart infusion broth, vitamin mixture, lactalbumin hydrolysate, casamino acids, α-ketoglutaric acid and yeast extract, one or more growth inhibitors for organisms other than Campylobacter selected from the group consisting of bile salts, Irgasan DP 300, sodium cefsulodin hydrate, cycloheximide and sodium cefoperazone, and one or more ingredients for absorbing oxygen selected from the group hemin, sodium metabisulfite, sodium carbonate and sodium pyruvate.
